(19) United States
(12) Patent Application Publication
(10) Pub. No.: US 2008/0275543 A1
(43) Pub. Date: Nov. 6, 2008

(54) Stent

(75) Inventors: Jason T. LENZ, Maplewood, MN (US); Jaykeep Y. Kokate, Maple Grove, MN (US); Matthew J. Miller, Stillwater, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/743,330

(22) Filed: May 2, 2007

(51) Int. Cl. A61F 2/82

(52) U.S. Cl. 623/1.42

(57) ABSTRACT

In at least one embodiment, the invention is directed to mechanisms that affect the elution rate of a therapeutic agent that has been deposited on the surface of at least a portion of a stent. Mechanisms include grooves formed in the therapeutic agent that is coating at least a portion of the surface of the stent.

In at least one embodiment, the invention is directed to the directional release of a therapeutic agent contained within a reservoir formed in at least one member of a stent.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to mechanisms that affect the elution rate of a therapeutic agent that has been deposited on the surface of at least a portion of a stent. Mechanisms include grooves formed in the therapeutic agent that is coating at least a portion of the surface of the stent.

In at least one embodiment, the invention is directed to the directional release of a therapeutic agent contained within a reservoir formed in at least one member of a stent.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a perspective view of a stent comprising a plurality of members.

FIG. 2 is a cross-sectional view of a portion of a member of the stent in FIG. 1.

FIG. 3 is the cross-sectional view of the member in FIG. 2 with micro grooves in the coating of therapeutic agent.

FIG. 4 is the cross-sectional view of the member in FIG. 2 with holes extending from the surface of the coating of therapeutic agent down to the member.

FIG. 5 is the cross-sectional view of the member in FIG. 2 with holes extending from the surface of the coating of therapeutic agent partially through the coating of therapeutic agent.

FIG. 6 is a cross-sectional view of a member with a reservoir in the body of the member and laser perforations extend from the surface of the member to the reservoir provide directional release of the substance deposited in the reservoir.

FIG. 7 is a cross-section view of a member with a reservoir in the body of the member, a metallic coating overlaying the reservoir and laser perforations in the metallic coating allow the direction release of the substance deposited in the reservoir.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

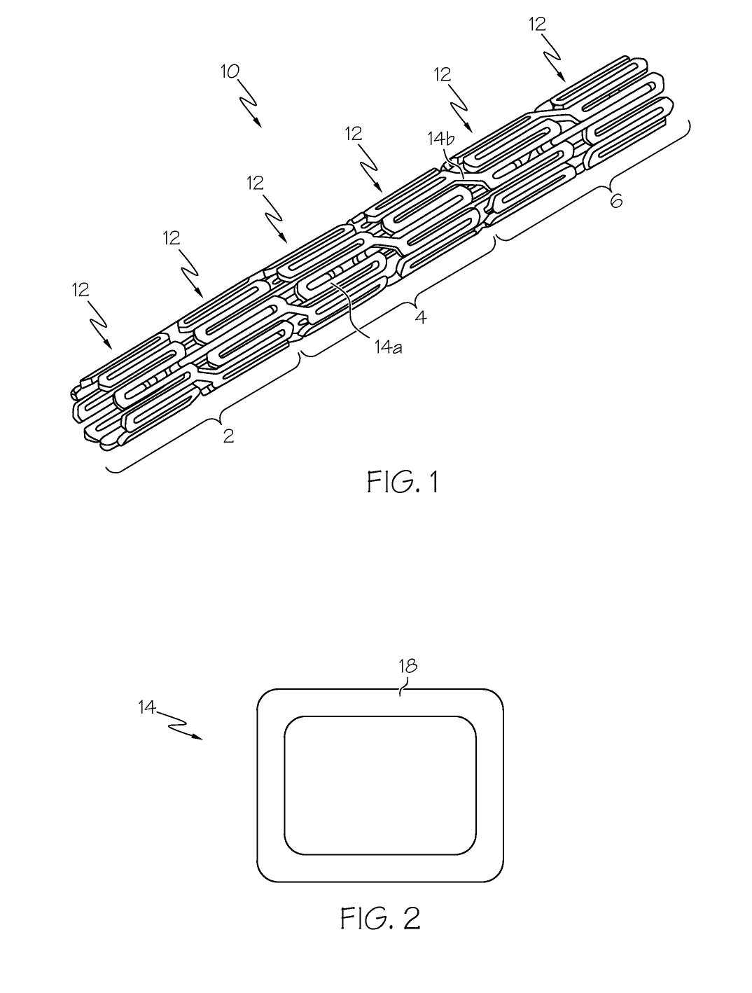

FIG. 1 depicts a stent 10 comprising a plurality of members 14 that form circumferential rings 11 extending about the circumference of the stent 10. As used in this application, members 14 include struts 14a and connectors 14b. The stent 10 illustrated in FIG. 1 is presented as an example of a configuration for a stent 10, as the present invention may be used with any stent 10 configuration desired. It is also within the scope of the invention for the stent 10 to have a braided tubular wall formed from at least two members. In this embodiment, the at least two members 14 may be wires with a round cross-section or ribbons with a width and thickness. As used in this application, an oblique angle is any angle between 0 and 180 degrees and includes 90 degrees.

To simplify the illustration of embodiments of the invention, the FIGS. 2-7 are a cross-section of one of the members 14 of the stent 10 in FIG. 1. Each member 14 has four sides from which therapeutic agents 18 can be eluted: the abluminal side (side of member 14 adjacent to the lumen wall), the luminal side (side of member 14 adjacent to the lumen) and the other two sides of the member 14 which are at an oblique angle to the luminal and abluminal sides.

A therapeutic agent 18 may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent 18 includes cellular material, the cellular material may include but is not limited to: cells of human origin and/or non-human origin as well as their respective components and/or derivatives thereof. Where the therapeutic agent 18 includes a polymer agent, the polymer agent may be a polystyrene-polyisobutylene-polystyrene triblock copolymer (SIBS), polyethylene oxide, silicone rubber and/or any other suitable substrate. Commonly assigned US Application Publication No 2006/0045901, hereby incorporated by reference in its entirety, contains a more extensive list of therapeutic agents 18 that may be used with the present invention.

In the embodiments, illustrated in FIGS. 2-5, the member 14 has a therapeutic agent 18 disposed on all the surfaces of the member 14. It is within the scope of the invention for the therapeutic agent 18 to be deposited on at least one surface of the member 14. In at least one embodiment, the therapeutic agent 18 is deposited on the luminal side of the member 14. In at least one embodiment, the therapeutic agent 18 is deposited on the abluminal side of the member 14. In at least one embodiment, the therapeutic agent 18 is deposited on both the luminal and abluminal sides of the member 14. If the member 14 has a round cross-section, it is within the scope of the invention for the therapeutic agent 18 to be deposited on only a portion of the circumference of the member 14.

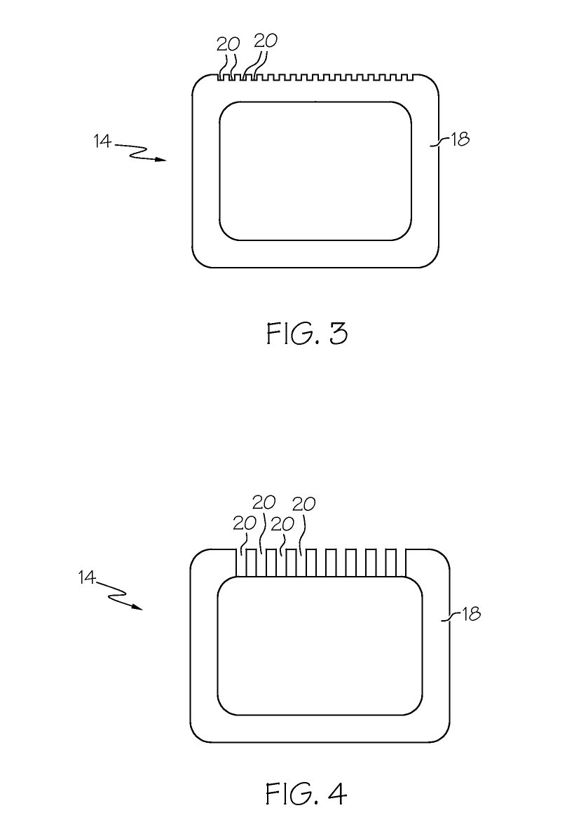

The coating of therapeutic agent 18 can have any depth or thickness and is deposited onto the surfaces of the member 14 by any means known in the art. As shown, for example in FIGS. 3-5, at least one surface of the coating of therapeutic agent 18 has at least one depression 20. It is within the scope of the invention for there to be one, two, three, four, five, six, seven, eight, nine, ten, eleven, twelve, thirteen, fourteen, fifteen, sixteen, seventeen, eighteen, nineteen, twenty or more depressions 20. The at least one depression 20, can have any shape, for example, but not limited to, grooves, channels, holes, and wells. The depression 20 is at an oblique angle to the surface of the therapeutic agent 18, as shown, for example, in FIG. 3. As used in this application, an oblique angle is any angle between 0 and 180 degrees and includes 90 degrees. In at least one embodiment, the depression 20 in the therapeutic agent 18 promotes healing at the implantation site. In at least one embodiment, the depression 20 in the therapeutic agent 18 affects the elution rate of the therapeutic agent 18.

In at least one embodiment a laser is used to make at least one depression 20 in the at least one coating of therapeutic agent 18. It is within the scope of the invention for any laser to be used. In at least one embodiment, crimping the coated stent 10 onto a forming mandrel at an elevated temperature forms at least one depression 20 into the coating of therapeutic agent 18.

The depressions 20 formed in the coating of therapeutic agent 18 can have any depth, as shown in FIGS. 3-5. Although the depressions 20 in FIGS. 3-5 have the same depth, it is within the scope of the invention for the depressions 20 formed in the therapeutic agent 18 on the surface of a member 14 to have different depths. The maximum depth of the depressions 20 is equal to the depth of the coating of therapeutic agent 18, as shown in FIG. 4. A depression 20 affects the elution rate of the therapeutic agent 18 because the depression 20 increases the surface area from which the therapeutic agent 18 elutes. Thus, the number and the surface area of depressions 20 can be adjusted to yield the desired surface area and therefore the elution rate of the therapeutic agent 18.

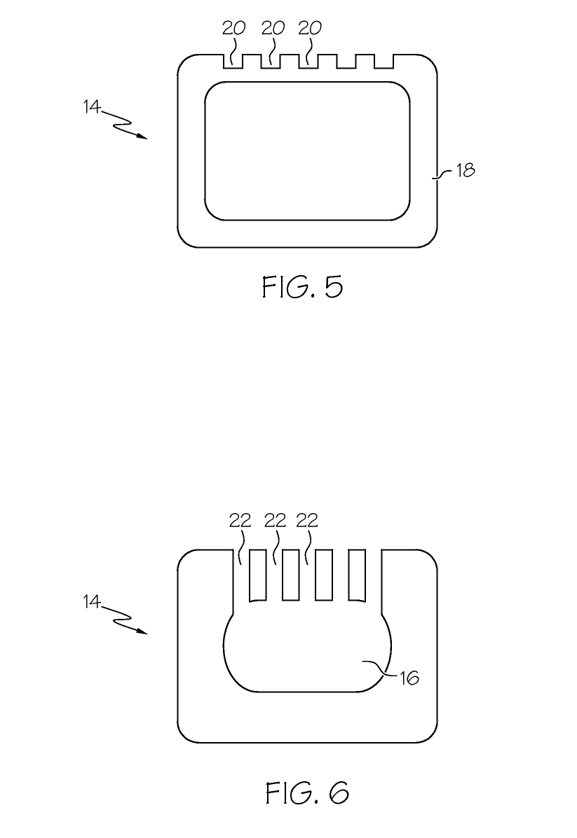

The depth of the depression 20 determines how much the depression 20 increases the surface area of the therapeutic agent 18. The depressions 20 in the therapeutic agent 18 in FIG. 5 have a greater surface area than the depressions 20 in the therapeutic agent 18 in FIG. 3 because the depressions 20 in FIG. 5 have a greater depth than the depressions 20 in FIG. 3. Note that although the bottom surface of the depression 20 is a part of the total surface area it does not contribute to the increase in surface area generated by the formation of the depression 20. This is due to the fact that the bottom surface of the depression 20 is a part of the original surface area of the therapeutic agent 18. Formation of the depression 20 only changed the position of that portion of the surface of the therapeutic agent 18. Thus, in FIG. 4, where the depressions 20 have a depth equal to the coating of therapeutic agent 18, the sides of the depressions 20 increase the total surface area while the bottom surfaces of the depressions 20 decrease the total surface area because the bottom surfaces do not have any therapeutic agent 18 to elute. The bottom surfaces of the depressions 20 in FIG. 4 are the surface of the member 14.

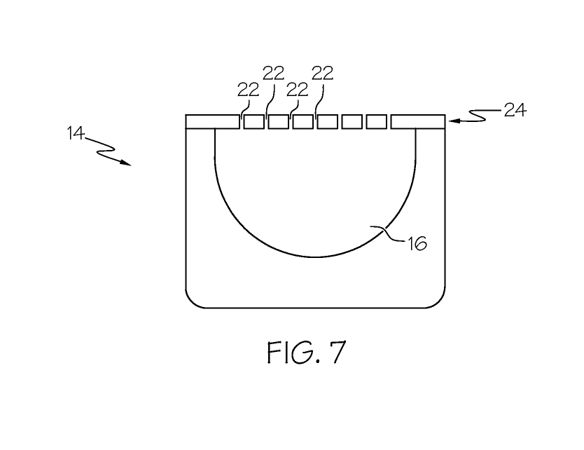

Therapeutic agent 18 may be deposited in a reservoir positioned within the body of the member 14 instead onto the surface of the member 14. FIG. 6 is an illustration of a reservoir 16 positioned within the body of a member 14 of the stent 10. FIG. 7 is an illustration of a member 14 that has a reservoir 16 that has been created on a surface of the member 14. In this embodiment, a coating 24 is applied to the member 14 after therapeutic agent 18 has been deposited into the reservoir 16 thereby covering the reservoir 16 and therapeutic agent 18. Examples of materials that can be used for the coating 24 include, but are not limited to, aluminum, aluminum oxide, magnesium, magnesium oxide, iron, iron oxide, tantalum, tantalum oxide, titanium, titanium oxide, tungsten, tungsten oxide, and ceramic materials. In at least one embodiment, the material used for the coating 24 is applied to the member 14 by a plasma deposition process. Note that if the coating 24 material is not oxidized prior to being deposited onto the member 14, the coating 24 material can be oxidized after it has been deposited onto the member 14.

Both reservoir 16 embodiments require a means by which the therapeutic agent 18 elutes from the reservoir 16. In at least one embodiment, a laser is used to drill holes 22 that form a passageway leading from the reservoir 16 to at least one surface of the member 14. Any laser may be used to drill the holes 22. If holes 22 are drilled on only one surface of the member 14, there is a directional release of therapeutic agent 18 from the reservoir 16. Thus, the therapeutic agent 18 can be targeted towards, for example, the luminal side of the member 14.

The elution rate of therapeutic agent 18 from the reservoir 16 is affected by the number of holes 22 and the size of the passageway of the holes 22. One way to increase the elution rate is to increase the number of holes 22. Another way to increase the elution rate is to increase the size of the passageway of the holes 22. In one embodiment, the laser produces holes 22 that are nano sized. In one embodiment, the laser produces holes 22 that are micro sized. Note that although the openings and passageway of the holes 22 can have any configuration, for example, but not limited to, round, square, rectangular, oval, oblong, bow-tie shaped, X-shaped, polygonal, irregular, and any combination thereof. The passageway of the hole 22 can have the same configuration as the opening, a different configuration from the openings of the hole 22 or more than one configuration.

Stents 10 have different regions and/or subregions. As a non-limiting example, the stent 10 in FIG. 1 can be divided into a proximal region 2, a middle region 4 and a distal region 6, where each region has two circumferential rings 11 of members 14. One of ordinary skill in the art will recognize that there are numerous ways in which the stent 10 of FIG. 1 can be designed to have different regions and/or subregions that have different sizes and positions along the longitudinal length of the stent 10.

As discussed in greater detail in U.S. patent application entitled Bifurcated Stent with Drug Wells for Specific Ostial, Carina, and Side Branch Treatment, Attorney Docket Number S63.2B-13099-US01, with inventors Dan Gregorich, Mike Meyer and Dave Friesen, hereby incorporated by reference herein in its entirety, a stent 10 can be used to deliver multiple therapeutic regimens from different regions and/or subregions of the stent 10. Examples of different regions of a stent (bifurcated and non-bifurcated) include, but are not limited to, the luminal side, the abluminal side, the proximal region (2), the distal region (6), the middle region (4), the main body of a bifurcated stent, the contralateral region of a bifurcated stent, the side branch of a bifurcated stent, members forming the side branch, and the perimeter member of a bifurcated stent.

As discussed above, the presence of depressions 20 in the therapeutic agent 18 affects the elution rate of the therapeutic agent 18. Similarly, the number and size of holes 22 from a reservoir 16 to the surface of the member 14 affects the elution rate of the therapeutic agent 18 from the reservoir 16. In at least one embodiment, at least one region of the stent 10 has a different elution rate of therapeutic agent 18 than at least one other region of the stent 10.

The following numbered statements characterize embodiments described above:

1. A method for manufacturing a reservoir in a member of a stent, comprising the steps of:

providing a stent, the stent having a plurality of members, each of the plurality of members having a first surface;

providing a laser;

directing the laser to the first surface; and

forming a reservoir in the first surface with the laser.

2. The method of statement 1, further comprising the steps of:

providing a therapeutic agent; and

depositing the therapeutic agent in the reservoir.

3. The method of statement 2 further comprising the step of:

depositing a coating of material over the reservoir, wherein the material is selected from at least one member of the group consisting of metal, metal oxides and ceramic materials.

4. The method of statement 3, the coating of material being deposited over the reservoir by plasma deposition.

5. The method of statement 3, further comprising the step of using the laser to bore at least one hole through the coating of material so that the hole extends from the first surface of the member to the reservoir, thereby providing a passageway from the reservoir to the first surface of the member.

6. The method of statement 3, the material being metal, wherein the metal is selected from at least one member of the group consisting of aluminium, magnesium, iron, tantalum, titanium, tungsten, and tungsten oxide, further comprising the step of oxidizing the coating of metal.

7. The method of statement 6, further comprising the step of using the laser to bore at least one hole through the coating of metal so that the hole extends from the first surface of the member to the reservoir, thereby providing a passageway from the reservoir to the first surface of the member.

8. A stent, the stent comprising a plurality of members, each of the plurality of members having a first surface and a body, the body having a volume, the body of at least one of the plurality of members defining a reservoir and at least one hole, the at least one hole extending from the reservoir to the first surface.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent, the stent comprising a plurality of members, at least one of the plurality of members having a first surface, the first surface having a coating deposited thereon, the coating comprising a first therapeutic agent, the coating defining at least one depression.

2. The stent of claim 1, the at least one depression having a configuration, the configuration selected from at least one member of the group consisting of grooves, channels, holes, wells, and any combination thereof.

3. The stent of claim 1, the coating having a thickness, the at least one depression having a depth, the depth of the at least one depression being at most equal to the thickness of the coating.

4. The stent of claim 1, the coating having a length, the at least one depression having a length, the length of the at least one depression being at most equal to the length of the coating.

5. The stent of claim 1, each of the plurality of members having a luminal side, the first surface being the luminal side.

6. The stent of claim 1, the plurality of members comprising at least two struts and at least one connector, the at least two struts forming at least two circumferential rings, the at least two circumferential rings being engaged by the at least one connector.

7. The stent of claim 6, wherein the at least one of the plurality of members having a first surface is selected from at least one member of the group consisting of the at least two struts, the at least one connector and any combination thereof.

8. The stent of claim 7, all of the at least one of the plurality of members having a first surface.

9. The stent of claim 1, the stent having a braided tubular wall, the plurality of members being interwoven to form the braided tubular wall.

10. The stent of claim 1, the plurality of members having a round cross-section and a circumference, the first surface being at least a portion of the circumference.

11. A stent, the stent having a body comprising a plurality of members, the plurality of members defining the body of the stent, the body comprising a first region and a second region, the first region having a first therapeutic agent, the second region having a second therapeutic agent, the first therapeutic agent having a first elution rate and the second therapeutic agent having a second elution rate, the first elution rate greater than the second elution rate.

12. The stent of claim 11, wherein the first region is selected from at least one member of the group consisting of the luminal side, the abluminal side, the proximal region, the distal region, the middle region, the main body of a bifurcated stent, the contralateral region, the side branch of a bifurcated stent, members forming the side branch, the perimeter member and any combination thereof.

13. The stent of claim 12, wherein the second region is selected from at least one member of the group consisting of the luminal side, the abluminal side, the proximal region, the distal region, the middle region, the main body of a bifurcated stent, the contralateral region, the side branch of a bifurcated stent, members forming the side branch, the perimeter member and any combination thereof, the second region being different than the first region.

14. A method of increasing the surface area of a coating on at least one member of a stent, comprising the steps of:
providing a stent, the stent comprising a plurality of members, each of the plurality of members having a first surface, at least one of the plurality of members having a coating, the coating deposited on the first surface, the coating having a first surface area; and
forming at least one depression in the coating, the at least one depression causing the coating to have a second surface area, the second surface area greater than the first surface area.

15. The method of claim 14, a laser being used for forming at least one depression in the coating.

16. The method of claim 14, a crimper being used for forming at least one depression in the coating.
